(19) United States
(12) Patent Application Publication
(10) Pub. No.: US 2022/0188940 A1
(43) Pub. Date: Jun. 16, 2022

(54) SYSTEM AND METHOD FOR REGULATING A VALUE OF A CRYPTOCURRENCY USED IN A HEALTH CARE NETWORK

(71) Applicant: Patientory, Inc., Atlanta, GA (US)

(72) Inventor: Chrissa Tanelia McFarlane, Atlanta, GA (US)

(21) Appl. No.: 17/607,221

(22) PCT Filed: Jun. 10, 2019
(86) PCT No.: PCT/US2019/036418

§ 371 Date: Oct. 28, 2021

Related U.S. Application Data
(60) Provisional application No. 62/683,513, filed on Jun. 11, 2018.
(60) Provisional application No. 62/683,524, filed on Jun. 11, 2018.
(60) Provisional application No. 62/683,537, filed on Jun. 11, 2018.
(60) Provisional application No. 62/683,556, filed on Jun. 11, 2018.
(60) Provisional application No. 62/683,568, filed on Jun. 11, 2018.

(51) Int. Cl. G06Q 40/00; G06Q 40/08; G06Q 20/36

(52) U.S. Cl. CPC G06Q 40/12; G06Q 20/367; G06Q 40/08

(57) ABSTRACT

A system and a method for regulating a value of a cryptocurrency used in a health care network are disclosed. The method includes providing a plurality of users connected to the health care network, a plurality of service providers connected to the health care network, a financial platform responsible for regulating the cryptocurrency, and a skill database utilizing machine learning for identifying and storing skills. A correlation coefficient may be determined between a transaction rate and a data event. The transaction rate corresponds to transactions made using the cryptocurrency, and the data event include at least one of usage of a skill by a service provider, addition of a new skill to the skill database, doctor density in an area, and usage of an emergency care. The financial platform may be notified for updating the value of the cryptocurrency based on the correlation coefficient.

### CROSS REFERENCE TO RELATED APPLICATIONS

The present disclosure is related and claims priority under 35 U.S.C. § 1.119(e) to U.S. provisional application Nos. 62/683,513, entitled SYSTEM AND METHOD FOR MANAGING PAYMENTS FOR ACCESSING PATIENTS INFORMATION; 62/683,524, entitled SYSTEM AND METHOD OF CONTROLLING ACCESS OF A USERS HEALTH INFORMATION STORED OVER A HEALTH CARE NETWORK; 62/683,537, entitled SYSTEM AND METHOD FOR REGULATING A VALUE OF A CRYPTOCURRENCY USED IN A HEALTH CARE NETWORK, 62/683,556, entitled SYSTEM AND METHOD FOR FACILITATING PAYMENT REQUESTS WITHIN A HEALTH CARE NETWORK, and 62/683,568, entitled SYSTEM AND METHOD OF MANAGING ACCESS OF A USERS HEALTH INFORMATION STORED OVER A HEALTH CARE NETWORK, all filed on Jun. 11, 2018, to Chrissa Tanelia McFarlane, the contents of all of which are hereby incorporated by reference in their entirety, for all purposes.

### FIELD OF THE DISCLOSURE

The present disclosure is generally related to cryptocurrency, and more particularly related to a method for regulating cryptocurrency value.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner The blocks are added to the blockchain in a linear and chronological order. One application of blockchain includes cryptocurrencies, which aim to avoid some of the fundamental problems associated with general currencies such as double usage. However, cryptocurrencies still present some obstacles for wider adoption.

### SUMMARY

In a first embodiment, a computer-implemented method for regulating a value of a cryptocurrency used in a healthcare network includes enabling the healthcare network to connect to a plurality of users and a plurality of service providers. The computer-implemented method also includes providing a financial platform responsible for regulating the cryptocurrency used in the healthcare network, and providing a skill database for identifying and storing skills. The computer-implemented method also includes determining a correlation coefficient between a transaction rate and a data event, wherein the transaction rate corresponds to transactions made using the cryptocurrency, and the data event includes at least one of usage of a skill by a service provider, addition of a new skill to the skill database, doctor density in an area, or usage of emergency care by at least a portion of the plurality of users, and notifying the financial platform for updating the value of the cryptocurrency based on the correlation coefficient.

In a second embodiment, a system for regulating a value of a cryptocurrency used in a healthcare network includes a memory storing instructions and one or more processors configured to execute the instructions. The instructions cause the system to enable the healthcare network to connect to a plurality of users and a plurality of service providers, to provide a financial platform responsible for regulating the cryptocurrency used in the healthcare network, and to provide a skill database for identifying and storing skills. The instructions also cause the system to determine a correlation coefficient between a transaction rate and a data event, wherein the transaction rate corresponds to transactions made using the cryptocurrency, and the data event includes at least one of usage of a skill by a service provider, addition of a new skill to the skill database, doctor density in an area, or usage of emergency care by at least a portion of the plurality of users and to notify the financial platform for updating the value of the cryptocurrency based on the correlation coefficient.

In yet other embodiment, a computer-implemented method for managing transactions in a healthcare network includes enabling the healthcare network to connect to a plurality of users and a plurality of service providers, and accessing a financial platform responsible for regulating a cryptocurrency used in the healthcare network. The computer-implemented method also includes accessing, via a blockchain network, a skill database for identifying and storing multiple skills relevant to the healthcare network, associating a cryptocurrency value with a data event based on an information from the financial platform, the data event involving a third party user accessing at least one of the skills from the skill database, notifying the financial platform for updating the cryptocurrency value based on the data event, and modifying a block string for the skill in the blockchain network, based on the data event.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a network connection diagram of a system, according to various embodiments.

FIG. 2A illustrates a method for symmetric encryption of data, according to various embodiments.

FIG. 2B illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in a health care network implemented, for example, over a blockchain network, according to various embodiments.

FIG. 6 illustrates a flowchart showing an example process carried out by a correlation module, according to various embodiments.

FIG. 7A illustrates an example correlation plot between a transaction rate of a cryptocurrency and doctor density, according to various embodiments.

FIG. 7B illustrates an example correlation plot between a transaction rate of a cryptocurrency and emergency care facilities, according to various embodiments.

FIG. 8 illustrates a flowchart showing an example process carried out by a valuation module, according to various embodiments.

FIG. 9 is a block diagram that illustrates a computer system used to perform at least some of the steps and methods in accordance with various embodiments.

### DETAILED DESCRIPTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, particular embodiments of the systems and methods will be described.

Current systems and methods for storing and managing transfer of health information between multiple parties in the healthcare system are often centralized structures subject to hacking, and yet mired in strict security regulations and onerous overhead costs. This state of affairs leads to a lack of efficient and transparent information exchange, to the ultimate detriment of patients and physicians Embodiments as disclosed herein resolve the above technical problem arising in the realm of healthcare data management by implementing a blockchain infrastructure to minimize security breaches and facilitate coordination between multiple entities and organizations, thus improving the health outcomes for patients.

In some embodiments, a blockchain infrastructure as disclosed herein allows the care providers to avoid medication errors, thus reducing the need for duplicate testing. Further, blockchain technology as disclosed herein effectively tracks and timestamps activities related to health information data. Thus, some embodiments provide a robust audit trail that ensures access to all interested and authorized parties to an updated version of a medical record.

Furthermore, some embodiments in a blockchain network as disclosed herein includes smart contracts configured with universal parameters. Accordingly, patients become the primary intermediaries for sending and receiving health information. Records stored in a blockchain network as disclosed herein are robust to tampering or error, and stored across multiple participating users (e.g., the entire blockchain network). Accordingly, recovery contingencies are unnecessary. Moreover, the transparency of a blockchain network as disclosed herein substantially reduces the number of data exchange integration points and the need for tedious reporting activities.

In some embodiments, a mobile application installed in client devices allow users to interact with the blockchain network and access features such as messaging, and access updated and accurate health information. Further, some embodiments provide tracking applications and other activity trackers to enable doctors, care providers, and other parties in the blockchain network to communicate on a single, easy to use platform. Furthermore, in some embodiments, artificial intelligence, machine learning, neural networks, and other nonlinear algorithms are incorporated to store and manage data in the blockchain network.

Some embodiments provide the ability for patients and other users of the blockchain network to access tokens from an external blockchain to convert into a supported cryptocurrency for access and use of storage features.

Embodiments of the present disclosure will be described more fully hereinafter with reference to the accompanying drawings in which like numerals may represent like elements throughout the several figures, and in which various example embodiments are shown. Embodiments may, however, be embodied in many different forms and should not be construed as limited to the embodiments set forth herein. The examples set forth herein are non-limiting examples and are merely examples among other possible examples.

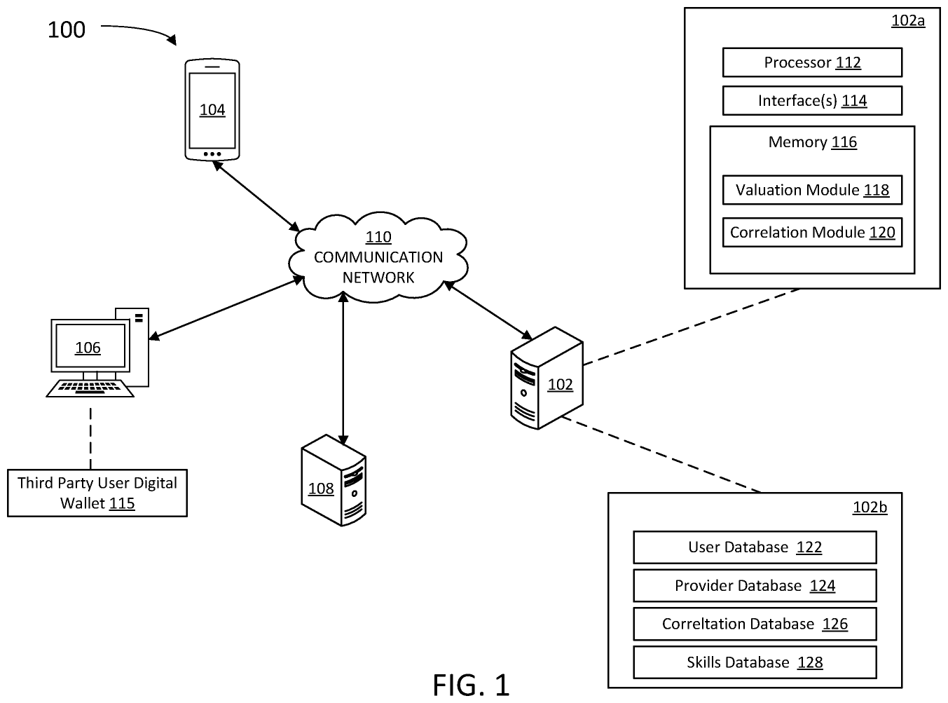

FIG. 1 illustrates a network connection diagram 100 of a Health Information Exchange (HIE) system 102 for regulating a value of a cryptocurrency used in a health care network. The HIE system 102 may include one or more user interfaces. The one or more user interfaces may be accessed by one or more users via one or more user devices 104. The HIE system 102 may be connected with a user device 104, a service provider device 106, and financial platform (e.g., coin market) 108 through a communication network 110.

The communication network 110 may be a wired and/or a wireless network. The communication network 110, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE system 102 may include a group of components 102a for regulating the value of the cryptocurrency used in the health care network. The group of components 102a may include a processor 112, interface(s) 114, a memory 116, a valuation module 118, and a correlation module 120.

The processor 112 may execute an algorithm stored in the memory 116 for regulating the value of the cryptocurrency used in the health care network. The processor 112 may also be configured to decode and execute any instructions received from one or more other electronic devices or server(s). The processor 112 may include one or more general-purpose processors (e.g., microprocessors) and/or one or more special purpose processors (e.g., digital signal processors (DSPs), System On Chips (SOCs), Field Programmable Gate Arrays (FPGAs), or Application-Specific Integrated Circuits (ASICs)). The processor 112 may be configured to execute one or more computer-readable program instructions, such as program instructions to carry out any of the functions described in this description.

The interface(s) 114 may help an operator to interact with the HIE system 102. The interface(s) 114 may either accept inputs from users or provide outputs to the users, or may perform both the actions. In one case, a user can interact with the interface(s) 114 using one or more user-interactive objects and devices. The user-interactive objects and devices may include user input buttons, switches, knobs, levers, keys, trackballs, touchpads, cameras, microphones, motion sensors, heat sensors, inertial sensors, touch sensors, or a combination of the above. Further, the interface(s) 114 may either be implemented as a Command Line Interface (CLI), a Graphical User Interface (GUI), a voice interface, or a web-based user-interface.

The memory 116 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 116 may include modules implemented as a program. In various embodiments, the memory 116 may include a valuation module 118 and a correlation module 120.

In various embodiments, several users may interact with the HIE system 102 using a user device 104. Although a single user device has been illustrated, several user devices could similarly be connected to the communication network 110. For example, a service provider may interact with HIE system 102 via a service provider device 106, associated with a third party user digital wallet 115. Further, each of the user devices may have a device ID. In various embodiments, the device ID may be a unique identification code such as an (International Mobile Equipment Identity) IMEI code or a product serial number. In some embodiments, a user may use a single user device or multiple user devices. Further, multiple users may use a single user device or multiple user devices. Further, the one or more users may receive and/or provide healthcare related products and services. The one or more users may include, for example, patients, family and friends of the patients, hospitals, physicians, nurses, specialists, pharmacies, medical laboratories, testing centers, insurance companies, or Emergency Medical Technician (EMT) services.

The user device 104 may be a stationary device, a portable device, or a device accessed remotely. The user device 104 may be, but is not limited to, a computer, a laptop, a tablet, a mobile phone, a smartphone, or a smart watch. In various embodiments, the user device 104 may include an imaging device that may be configured to capture a visual graphical element, the visual graphical element such as, but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In various embodiments, the imaging device may be located external to the user device 104. In various embodiments, the imaging device may be connected to the user device 104 wirelessly or via a cable. In some embodiments, image data of the visual graphical element may be transmitted to the user device 104 via the communication network 110.

In various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, the camera may be controlled by a processor natively embedded in the user device 104. In various embodiments, the imaging device may include a screen capturing software (for example, screenshot) that may be configured to capture and/or scan the QR code on a screen of the user device 104.

In various embodiments, a group of databases 102b may be connected to the HIE system 102. In one case, the group of databases 102b may be implemented over a blockchain network (such as a PTOYNet blockchain network or a PTOYNet Ethereum™ blockchain network), and may be present as different databases installed at different locations. The group of databases 102b may include a user database 122, a provider database 124, a correlation database 126, and a skills database 128. The group of databases 102b may be configured to store data belonging to different users and data for functioning of the HIE system 102. Different databases can be used in accordance with various embodiments; however, a single database may also be used for storing the data. Usage of the different databases may also allow segregated storage of different data and may thus reduce time to access desired data. In one case, the data may be encrypted, time-dependent, piece-wise, and may be present as subsets of data belonging to each user. For example, the data may represent the results of one medical test in a series of multiple medical tests.

In one embodiment, the group of databases 102b may operate collectively or individually. Further, the group of databases 102b may store data as tables, objects, or other data structures. Further, the group of databases 102b may be configured to store data retrieved or processed by the HIE system 102. The data may include, but is not limited to, a patient medical history, medical charts, medications, prescriptions, immunizations, test results, allergies, insurance provider(s), or billing information. Further, the data may be time-dependent and piece-wise. Further, the data may represent a subset of data for each patient. In an example, the data may represent results of a medical test in a series of multiple medical tests. Further, the data may be securely stored and the data may be encrypted.

In accordance with various embodiments, information stored in the group of databases 102b may be accessed based on users' identities and/or the users' authorities. The users' identities may be verified in one or more ways such as, but not limited to, biometric authentication (or bio-authentication), password or PIN information, user device registrations, a second-level authentication, or a third-level authentication. For example, the users' identities may be verified by the HIE system 102. Information provided by the users in real-time may be used, by the HIE system 102, to confirm the users' identities. For example, the users' identities may be verified using a name, a password, one or more security questions, or a combination thereof. For example, a user may be identified using an encryption key and/or a decryption key.

In accordance with various embodiments, the data stored in the group of databases 102b may be accessed at different levels, for example using a first level subsystem and a second level subsystem. For example, a user may directly access the first level subsystem. To access data stored in the second level subsystem, the second level subsystem may be accessed through the first level subsystem. In some embodiments, the communication between the first level subsystem and the second level subsystem may be encrypted. For example, the second level subsystem may be implemented over a blockchain network (such as a PTOYNet blockchain network). In various embodiments, the PTOYNet blockchain network may be used to implement smart contracts.

In an exemplary scenario, a primary care physician may input data into the HIE system 102 using the user device 104. The data may be processed by the first level subsystem and the second level subsystem. This may be done successively. The data may be stored on the first level subsystem and/or the second level subsystem of the HIE system 102. This may be done successively. The data may include, but is not limited to, one or more instructions to a patient to see a physician specialist. Further, the data may be stored in one or more blockchains of the second level subsystem. The patient may be able to access the data relating to the patient's care provided by the primary care physician. This may be done successively. The patient may be able to retrieve the data using the user device 104 of the patient. This may be done successively.

In accordance with various embodiments, the patient may communicate with the physician specialist using the HIE system 102. In some embodiments, the physician specialist may be able to access the data of the patient from the first level subsystem and/or the second level subsystem. Further, the physician specialist may be able to communicate with the patient. In some embodiments, all (or substantially all) communications between the primary care physician, the physician specialist, and the patient may be stored and may be accessible on a blockchain network (such as a PTOYNet blockchain network or a PTOYNet Ethereum™ blockchain network).

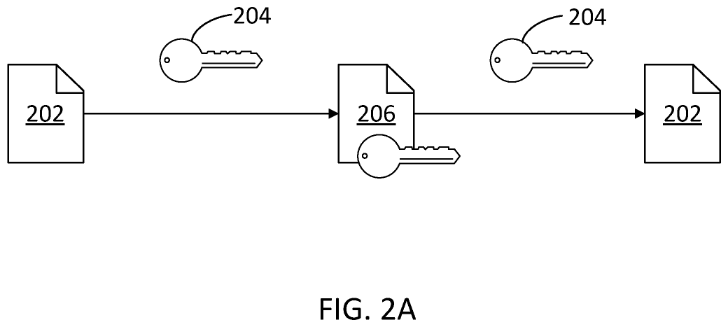

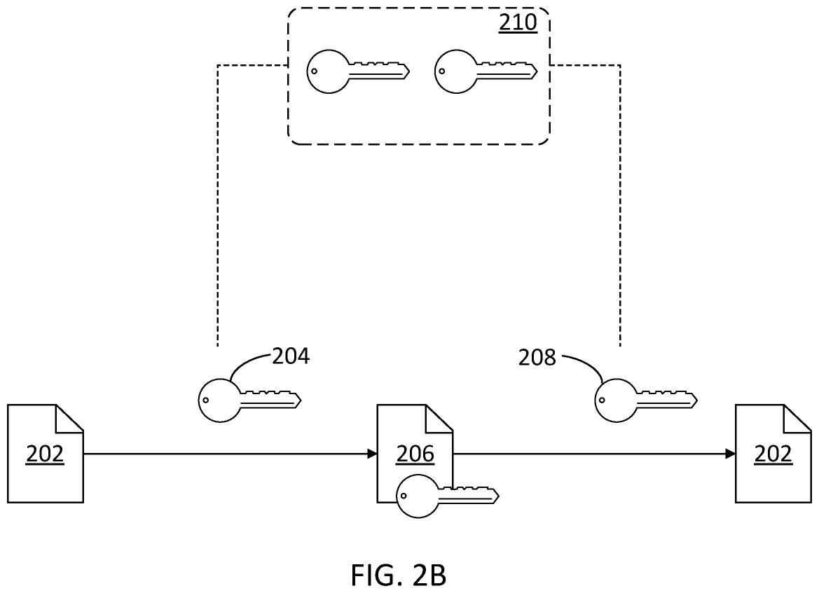

FIG. 2A illustrates a method for symmetric encryption of data, in accordance with various embodiments. Original data 202 may be encrypted using a key 204 to obtain an encrypted data 206. The encrypted data 206 may be decrypted using the key 204 to obtain back the original data 202. In some embodiments, encryption and decryption of the data may be performed using a same key. Further, one or more parties involved in a communication may have the same key to encrypt and decrypt the data.

FIG. 2B illustrates a method for asymmetric encryption of data, in accordance with various embodiments. Original data 202 may be encrypted using a key 204 to obtain encrypted data 206. The encrypted data 206 may be decrypted using another key 208 to obtain the original data 202. In some embodiments, encryption and decryption of the data may be performed using different keys, e.g., a key pair 210.

In some embodiments, the steps illustrated in FIGS. 2A-B may be initiated by users who generate a new profile on the blockchain network. Private keys may be stored in decentralized and distributed hashes through the blockchain network. In some embodiments, the steps illustrated in FIGS. 2A-B may be partially performed in either one of devices 104 and 106, in HIE system 102, or financial platform 108. For example, in some embodiments, HIE system 102 may install a software development kit (SDK) or a key generator application in user device 104 or in service provider device 106 to perform at least some of the steps illustrated in FIGS. 2A-B. Likewise, keys 204, 208, and key pair 210 may be stored in a memory of either one of device 104, service provider device 106, in HIE system 102, or in financial platform 108, or in an associated database (e.g., any one of databases 102b).

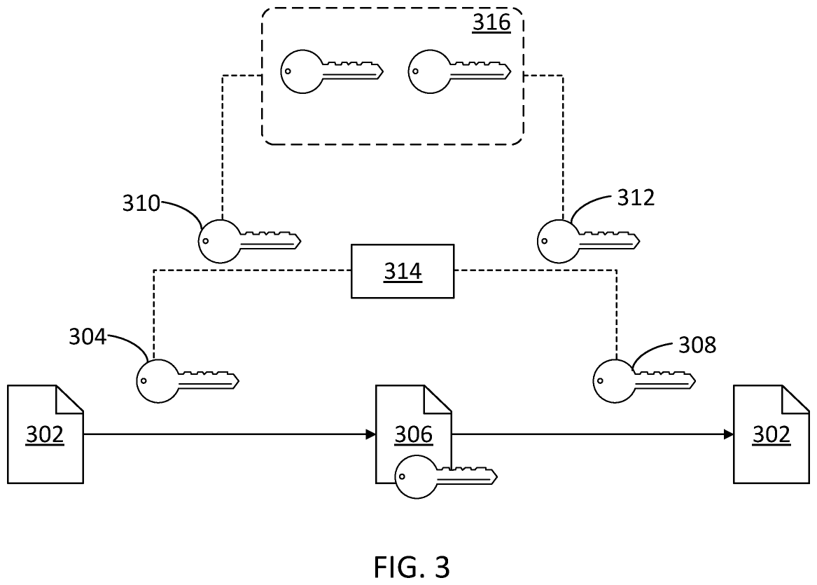

FIG. 3 illustrates a method for hybrid encryption of data, in accordance with various embodiments. Both symmetric encryption and asymmetric encryption techniques may be used in tandem. For example, the symmetric encryption technique may be used to encrypt data 302 using a symmetric key 304 for producing encrypted data 306. The encrypted data 306 may be decrypted using another symmetric key 308 for obtaining data 302. Further, a public key 310 may be used to encrypt the symmetric key 304 and a private key 312 may be used to encrypt the symmetric key 308, stored as an encrypted key 314. The public key 310 and the private key 312 may form a key pair 316.

In some embodiments, the steps illustrated in FIG. 3 may be initiated by users who generate a new profile on the blockchain network. Private keys may be stored in decentralized and distributed hashes through the blockchain network. In some embodiments, the steps illustrated in FIG. 3 may be partially performed in either one of devices 104 and 106, in HIE system 102, or financial platform 108. For example, in some embodiments, HIE system 102 may install a software development kit (SDK) or a key generator application in user device 104 or in service provider device 106 to perform at least some of the steps illustrated in FIG. 3. Likewise, keys 204, 208, and key pair 210 may be stored in a memory of either one of user device 104, service provider device 106, in HIE system 102, or in financial platform 108, or in an associated database (e.g., any one of databases 102b).

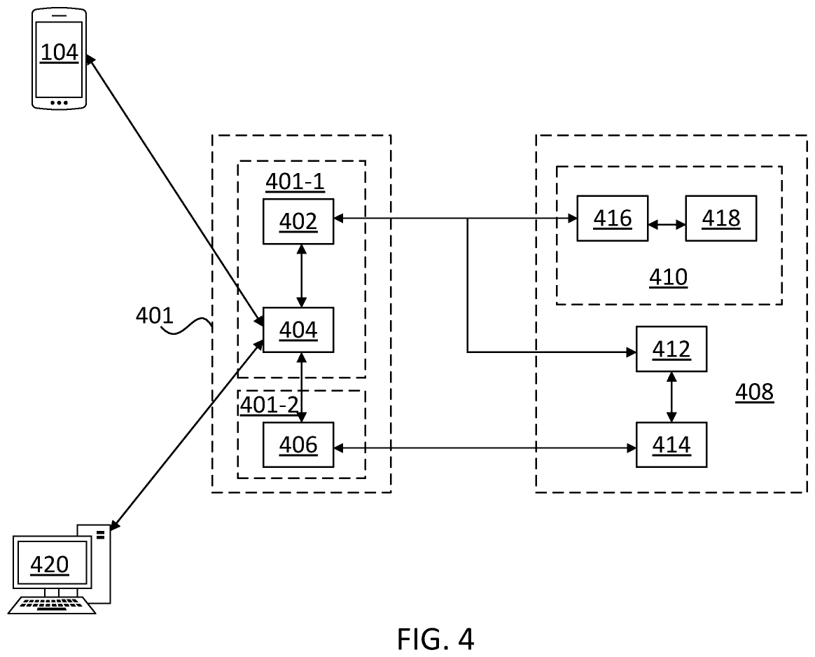

FIG. 4 illustrates a system 401 for storing and accessing data in a health care network, according to various embodiments. A first level subsystem 401-1 may include a core service component 402 and a Remote Procedure Call (RPC) component 404. A second level subsystem 401-2 may include a blockchain node 406. Blockchain node 406 may be a public node or a private node in a blockchain network having a layer over a public blockchain network, enabling the private node to perform private transactions via consensus algorithms (e.g., a Quorum blockchain node). In various embodiments, first level subsystem 401-1 may include the core service component 402, and second level subsystem 401-2 may include the RPC component 404 and the blockchain node 406. Further, the core service component 402 of first level subsystem 401-1 may be present in communication with third-party servers and databases of a hospital computing network 408. The hospital computing network 408 may include a file system module 410, an EHR synchronization service 412, and a blockchain node 414 (e.g., a Quorum blockchain node). Further, the file system module 410 may include a file system manager 416 and a file system node 418. The blockchain node 406 of second level subsystem 401-2 may communicate with the blockchain node 414 of the hospital computing network 408. Patients may access the health care network for storing data through the user device 104, and a representative of a hospital may access the health care network through another device 420 (e.g., a third party user through a desktop).

In accordance with various embodiments, the representative of the hospital may want to synchronize Electronic Health Record (EHR) data of a patient, e.g., by using corresponding blockchain hashes. Successively, first level subsystem 401-1 and second level subsystem 401-2 may ask the patient for permission to allow a representative of the hospital to store the EHR data of the patient, through the file system module 410. Based at least on the permission granted by the patient, a signed transaction may be created to confirm the permission of the hospital to store the EHR data. Further, the signed transaction may activate a smart contract that may add hospital identification information such as a blockchain address to a list of permitted users. In some embodiments, the signed transaction and the smart contract are stored in file system module 410.

Further, the signed transaction may be transmitted from the user device 102 to the RPC component 404 of the first level subsystem and/or the second level subsystem. The RPC component 404 may communicate the signed transaction to the blockchain node 406 of the second level subsystem. This may be done successively. The blockchain node 406 may activate one or more smart contracts. This may be done successively. Thereafter, the blockchain node 406 may revise a state of one or more blockchains.

Further, based at least on the permission granted by the patient, the EHR synchronization service may obtain a list of patients from the RPC component 404. Further, the EHR synchronization service may confirm whether the patient has granted permission. Based at least on the permission, the first level subsystem and the second level subsystem may obtain the EHR data and may calculate a hash function for the EHR data. The HIE system 102 may match the hash function of the EHR data with a hash function for the patient blockchain on the blockchain node 406 of the second level subsystem. This may be done successively. Thereafter, if the hash function of the EHR data matches with the hash function for the patient blockchain on the blockchain node 406 of the second level subsystem, the EHR data of the patient may remain unchanged.

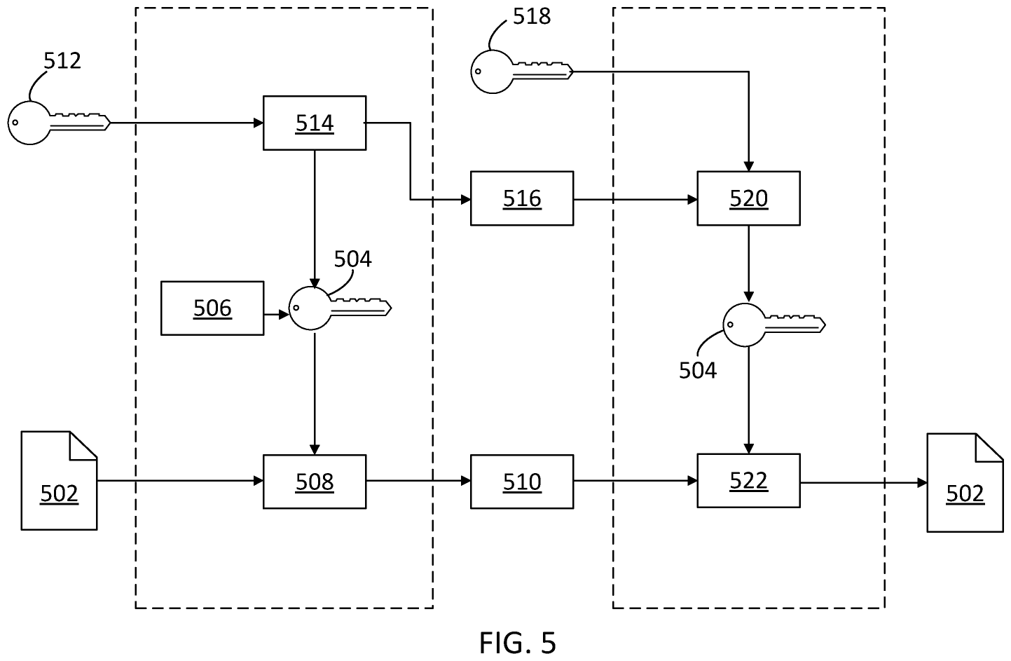

FIG. 5 illustrates an example of a system for storing and accessing data in a health care network, according to some embodiments (cf. FIGS. 1 and 4). The HIE system 102 may execute an application for determining permission from the user for obtaining EHR data 502. For example, if the user grants the permission, the HIE system 102 may obtain the EHR data 502 for calculating a hash function for the EHR data 502. The HIE system 102 may match the hash function of the EHR data 502 with a hash function for the user blockchain on the blockchain node of the second level sub-system. In various embodiments, if the two hash functions match, there is no change to the user's EHR data 502. In various embodiments, if the two hash functions do not match, the HIE system 102 may generate a random string, e.g., secret key 504, through a random key generator 506. The secret key 504 may be used for Advanced Encryption Standard (AES) encryption of the EHR data 502, in an AES encryptor 508, for generating encrypted EHR data 510.

In accordance with various embodiments, the secret key 504 may then be encrypted by, for example, a Rivest-Shamir-Adleman (RSA) public key 512 of the patient, in an RSA encryptor 514, to generate an encrypted secret key 516. The HIE system 102 may further send the encrypted EHR data 510 to the core service component 402 for forwarding the data to the file system manager 416 of the hospital computing network 408 for storage. Further, the file system manager 416 may send a file system hash function to the core service component 402 for further sending the file system hash function to EHR synchronization service 412. The EHR synchronization service 412 may further update the patient smart contract with the new file system hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to an RPC component 404 for granting permission to the hospital representative to view the EHR data 502. Once the permission is granted, the signed transaction may be added to the blockchain node 414 and a new smart contract will be created for a blockchain corresponding to the hospital representative. After adding the signed transaction, the hospital representative may be able to view the EHR data 502 of the user on a device.

In accordance with various embodiments, in order to view the EHR data 502 on the device, the HIE system 102 may collect the encrypted EHR data 510 from the user's blockchain and may decrypt the encrypted EHR data 510 using a patient's RSA private key 518. The HIE system 102 may decrypt the encrypted secret key 516, in an RSA decryptor 520, using an RSA private key of the hospital representative. The encrypted EHR data 510 may be decrypted using the RSA public key 512 of the hospital representative, in an AES decryptor 522. Further, the HIE system 102 may load the decrypted EHR data 502 to the smart contract previously created for the hospital representative.

After loading the decrypted EHR data 502, the RPC component 404 may obtain the signed transaction from the patient's user device and transmit the signed transaction to the blockchain node 406 of the second level subsystem. The blockchain node 406 may confirm ownership of the signed transaction and may execute the smart contract for the hospital representative to view the user's data.

In accordance with various embodiments, the patient may decline permission for the hospital representative to have access to the EHR data 502. In such a scenario, the user, through a user device, may send a signed transaction revoking permission to the RPC component 404. The RPC component 404 may forward the signed transaction to the blockchain node 406 of the second level subsystem. The blockchain node 406 may confirm ownership of the signed transaction and may delete the smart contract previously created to allow the hospital representative to have access to the patient's EHR data 502.

In accordance with various embodiments, the HIE system 102 may execute cryptocurrency valuation using a blockchain patient record. In various embodiments, the user may hold several authorities while purchasing a cryptocurrency unique value drivers specific to the blockchain platform. Such a scenario may help the user to acknowledge valuation fluctuations in a financial platform, e.g., a financial platform 108. After acknowledgment, the valuation fluctuations may be communicated with currency exchanges in which the cryptocurrency is traded. The communication may enable a publicly traded price to be accurate by incorporating unique value drivers that are specific to the blockchain platform.

Further, the HIE system 102 may include a health record network for an intermediary enabling sharing of user's medical records with providers. For enabling sharing, the user may grant specific permissions to the providers for accessing parts of the user's medical records stored in the user database 122 implemented over a blockchain network (such as a PTOYNet blockchain network or a PTOYNet Ethereum™ blockchain network). The user may also grant specific permissions to modify the user's medical records in the user database 122. In various embodiments, the user may include any users constituting a value chain, such as, for example, doctors, nurses, etc. In various embodiments, the user may be remote doctors logging into the HIE system 102 or doctors present in hospitals.

Further, the HIE system 102 may include a user database containing account information and activity of each user linked to the HIE system 102. In various embodiments, the account information and activity may include, for example, location, identifying information, and data relationships of the users with the providers. In some embodiments, the users added to the HIE system 102 can be in a direct relationship to the value of the system and hence value to the cryptocurrency.

Further, the HIE system 102 may also include a provider database 124 containing account information and activity of each provider in the system. In various embodiments, the providers may include, for example, hospitals, insurance companies, clinical research organization (CRO), drug companies, and the like. In some embodiments, the providers added to the HIE system 102 can be in a direct relationship to the value of the system and hence value to the cryptocurrency.

In accordance with various embodiments, the skills database 128 of the HIE system 102 may contain information related to best practices collated by a health record network that can be accessed by the providers. The information related to best practices may be related to the value of the cryptocurrency. In various embodiments, factors such as, for example, improved payment methods (e.g., machined learned), disease control (e.g., machined learned), and the like may also relate to value of the cryptocurrency. Further, the correlation database 126 of the HIE system 102 may contain information related to the correlation module 120. In various embodiments, the correlation database 126 may include information related to output of the correlation module 120.

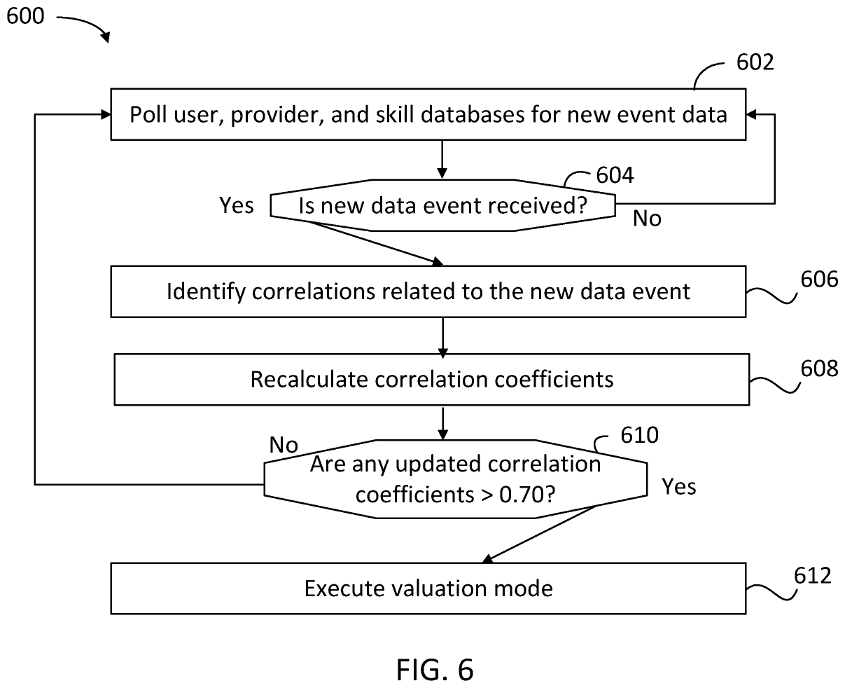

FIG. 6 illustrates an example process carried out by correlation module 120 in flowchart 600, according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the correlation module 120 may poll, for example, the user, provider, and skill databases for determining new data events such as, for example, a new enrollment, a new skill, or usage of the user or the provider, at step 602. Further, the correlation module 120 may check for receiving a new event, at step 604. In various embodiments, while a new data event is not yet received, the correlation module 120 may continue to poll for a new data event, back at step 602. In various embodiments, if new data events are received by the HIE system 102, the correlation module 120 may identify correlations related to the type of data event received, at step 606. For example, if the new data event is related to skill usage by the provider, the HIE system 102 may pull correlations related to skill usage.

Continuing, the retrieved correlations may be recalculated to include the value of the newest data point, at step 608. For example, once the correlations are updated, the correlation module 120 may determine if any updated correlation coefficients have value greater than, for example, about 0.70, at step 610. Where such correlation coefficients are identified, the correlation module 120 may execute the valuation module 118 at step 612. Otherwise, the polling for new data events may continue at step 602.

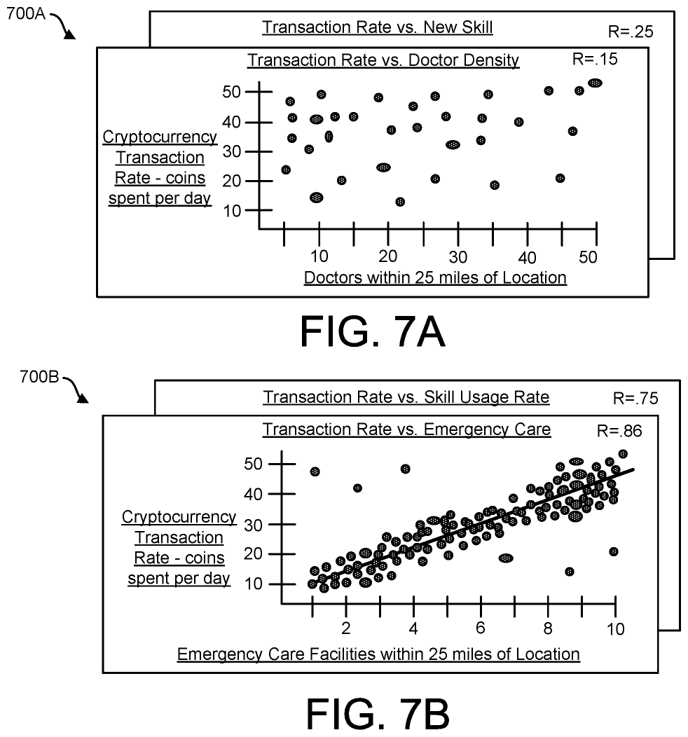

FIG. 7A illustrates an example correlation plot 700A between transaction rate of the cryptocurrency and doctor density, according to some embodiments. Such correlations may be run in an algorithm module (e.g., machine learning algorithm module) and may be stored in a database (e.g., machine learning database). The HIE system 102 may filter the data by the transaction rate of the system's cryptocurrency and find various correlations with the provider and user behavior and attributes. In some embodiments, a correlation plot may be developed between the transaction rate of the cryptocurrency and number of doctors present within a certain range of a location of a new data event (e.g., as determined by the smart contract), or about 25 miles in this example. In some embodiments, since the correlation coefficients of R=0.25 and R=0.15 may be determined to be below a threshold value, such as about 70%, the HIE system 102 may determine that there is a low or no correlation between the transaction rate vs. new skill and transaction rate vs. doctor density.

In various embodiments, the HIE system 102 may implement the correlation module when there is no correlation and no data points to be stored in the correlation database 126. In various embodiments, the HIE system 102 may implement the correlation module 120 when a large number of providers (when they are specifically doctors) does not contribute to a higher transaction rate. Such a scenario may occur, for example, during an emergency care situation, wherein the user base may not be incentivized to share their data with a large number of doctors.

FIG. 7B illustrates an example correlation plot 700B between transaction rate of the cryptocurrency and emergency care facilities, according to some embodiments. The HIE system 102 may filter the data by the transaction rate of the system's cryptocurrency and may find various correlations with the provider and user behavior and attributes. In some embodiments, the correlation plot is developed between the transaction rate and a number of emergency care facilities present within 25 miles, location of the new data event depicting a correlation coefficient of 86%, which may be determined to be above a predetermined threshold, such as a 70% threshold. Such a scenario may occur, for example, when, because there are more emergency care facilities present in a given area, there is a higher transaction rate of the cryptocurrency as there are more instances of enrolled providers. Therefore, the data points may be stored in the correlation database 126.

Further, the correlation module 120 may identify changes in values of number of users and providers. In various embodiments, the correlation module 120 may also identify skills that are highly correlated to shifts in demand (and therefore exchange value) for the system's cryptocurrency. Further, the correlation module 120 may act as a correlation engine that can include a software application for programmatically understanding relationships. In various embodiments, the correlation engine may also be used in systems management tools to aggregate, normalize, and analyze event log data using predictive analytics and fuzzy logic to alert the systems administrator during the time of a problem.

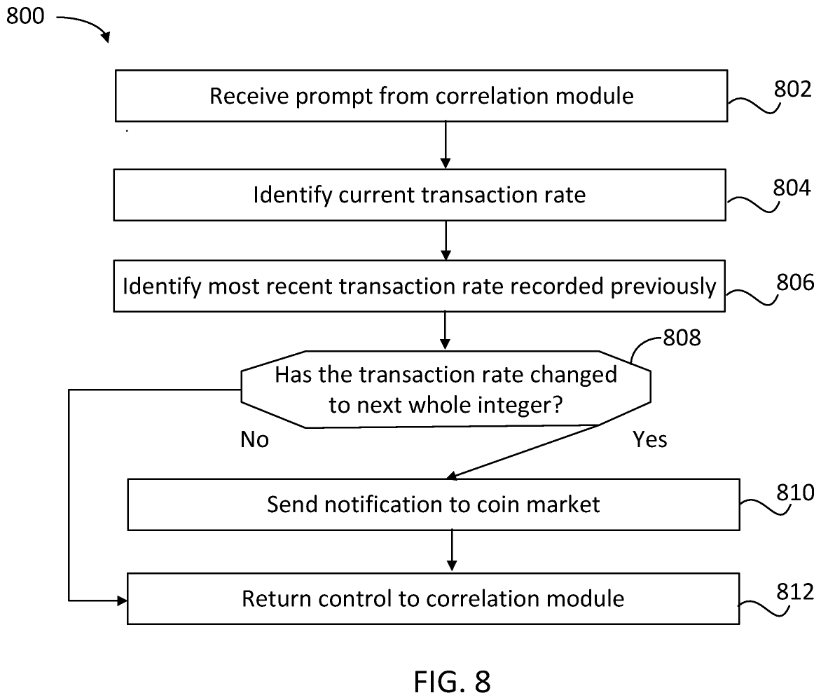

FIG. 8 illustrates an example process carried out by valuation module 118 in flowchart 800, according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The valuation module 118 may be invoked when a prompt is received, from the correlation module 120, of a correlation with a coefficient greater than a particular threshold, such as 0.70, at step 802. Further, the valuation module 118 may identify current currency transaction rate associated with the correlated data point, at step 804.

Further, the valuation module 118 may identify the most recent transaction rate previously recorded, at step 806. The valuation module 118 may determine whether the transaction rate has changed to meet a threshold increase. While step 808 in FIG. 8 uses the next whole integer, e.g., from 1.9 to 2.0, it should be understood that any threshold increase can be used, including fractional increases. While the currency transaction rate has crossed such a threshold increase, a notification may be sent to a coin market (or financial platform), at step 810. In one case, the notification is sent to the financial platform 108 so that trading public has accurate information with which to value the cryptocurrency. While the currency transaction rate has not crossed an integer threshold or after the notification is delivered, the valuation module 118 may return control to the correlation module 120, at step 812.

### Computer System

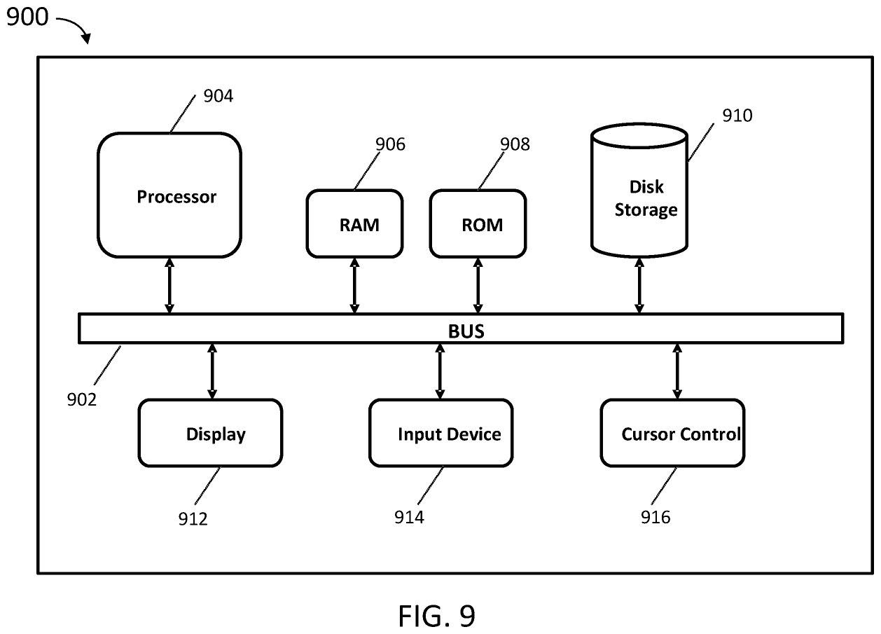

FIG. 9 is a block diagram that illustrates a computer system 900, upon which embodiments, or portions of the embodiments, of the present teachings may be implemented. In various embodiments of the present teachings, computer system 900 can include a bus 902 or other communication mechanism for communicating information, and a processor 904 coupled with bus 902 for processing information. In various embodiments, computer system 900 can also include a memory 906, which can be a random access memory (RAM) or other dynamic storage device, coupled to bus 902 for determining instructions to be executed by processor 904. Memory 906 also can be used for storing temporary variables or other intermediate information during execution of instructions to be executed by processor 904. In various embodiments, computer system 900 can further include a read-only memory (ROM) 908 or other static storage device coupled to bus 902 for storing static information and instructions for processor 904. A storage device 910, such as a magnetic disk or optical disk, can be provided and coupled to bus 902 for storing information and instructions.

In various embodiments, computer system 900 can be coupled via bus 902 to a display 912, such as a cathode ray tube (CRT) or liquid crystal display (LCD), for displaying information to a computer user. An input device 914, including alphanumeric and other keys, can be coupled to bus 902 for communicating information and command selections to processor 904. Another type of user input device is a cursor control 916, such as a mouse, a trackball, or cursor direction keys for communicating direction information and command selections to processor 904 and for controlling cursor movement on display 912. This input device 914 typically has two degrees of freedom in two axes, a first axis (e.g., x) and a second axis (e.g., y), that allows the device to specify positions in a plane. However, it should be understood that input devices 914 allowing for 3-dimensional (x, y, and z) cursor movement are also contemplated herein.

Consistent with certain implementations of the present teachings, results can be provided by computer system 900 in response to processor 904 executing one or more sequences of one or more instructions contained in memory 906. Such instructions can be read into memory 906 from another computer-readable medium or computer-readable storage medium, such as storage device 910. Execution of the sequences of instructions contained in memory 906 can cause processor 904 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 904 for execution. Such a medium can take many forms, including but not limited to, non-volatile media, volatile media, and transmission media. Examples of non-volatile media can include, but are not limited to, optical, solid state, and magnetic disks, such as storage device 910. Examples of volatile media can include, but are not limited to, dynamic memory, such as memory 906. Examples of transmission media can include, but are not limited to, coaxial cables, copper wire, and fiber optics, including the wires that include bus 902.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to a computer-readable medium, instructions or data can be provided as signals on transmission media included in a communications apparatus or system to provide sequences of one or more instructions to processor 904 of computer system 900 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein including flow charts, diagrams, and the accompanying disclosure can be implemented using computer system 900 as a standalone device or on a distributed network of shared computer processing resources such as a cloud computing network.

In accordance with various embodiments, the systems and methods described herein can be implemented using computer system 900 as a standalone device or on a distributed network of shared computer processing resources such as a cloud computing network. As such, a non-transitory computer-readable medium can be provided in which a program is stored for causing a computer to perform the disclosed methods for identifying mutually incompatible gene pairs.

It should also be understood that the preceding embodiments can be provided, in whole or in part, as a system of components integrated to perform the methods described. For example, in accordance with various embodiments, the methods described herein can be provided as a system of components or stations for analytically determining novelty responses.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, their specific configuration, communication, and physical orientation with respect to each other. One skilled in the art should readily appreciate that these components can have various configurations and physical orientations (e.g., wholly separate components, units, and subunits of groups of components, different communication regimes between components).

Embodiments disclosed herein include:

A. A computer-implemented method for regulating a value of a cryptocurrency used in a healthcare network includes enabling the healthcare network to connect to a plurality of users and a plurality of service providers. The computer-implemented method also includes providing a financial platform responsible for regulating the cryptocurrency used in the healthcare network, and providing a skill database for identifying and storing skills. The computer-implemented method also includes determining a correlation coefficient between a transaction rate and a data event, wherein the transaction rate corresponds to transactions made using the cryptocurrency, and the data event includes at least one of usage of a skill by a service provider, addition of a new skill to the skill database, doctor density in an area, or usage of emergency care by at least a portion of the plurality of users, and notifying the financial platform for updating the value of the cryptocurrency based on the correlation coefficient.

B. A system for regulating a value of a cryptocurrency used in a healthcare network includes a memory storing instructions and one or more processors configured to execute the instructions. The instructions cause the system to enable the healthcare network to connect to a plurality of users and a plurality of service providers, to provide a financial platform responsible for regulating the cryptocurrency used in the healthcare network, and to provide a skill database for identifying and storing skills. The instructions also cause the system to determine a correlation coefficient between a transaction rate and a data event, wherein the transaction rate corresponds to transactions made using the cryptocurrency, and the data event includes at least one of usage of a skill by a service provider, addition of a new skill to the skill database, doctor density in an area, or usage of emergency care by at least a portion of the plurality of users and to notify the financial platform for updating the value of the cryptocurrency based on the correlation coefficient.

C. A computer-implemented method for managing transactions in a healthcare network includes enabling the healthcare network to connect to a plurality of users and a plurality of service providers, and accessing a financial platform responsible for regulating a cryptocurrency used in the healthcare network. The computer-implemented method also includes accessing, via a blockchain network, a skill database for identifying and storing multiple skills relevant to the healthcare network, associating a cryptocurrency value with a data event based on an information from the financial platform, the data event involving a third party user accessing at least one of the skills from the skill database, notifying the financial platform for updating the cryptocurrency value based on the data event, and modifying a block string for the skill in the blockchain network, based on the data event.

Each of embodiments A, B, and C may have one or more of the following additional elements in any combination: Element 1, wherein the service providers include hospitals, insurance companies, Contract Research Organizations (CROs), and drug companies, further including adding a new service provider to the healthcare network based on a correlation between the service provider and the value of the cryptocurrency. Element 2, wherein the skills are machine learned, further including adding a new skill based on a correlation between the new skill and the value of the cryptocurrency. Element 3, wherein the skills include a best practice, improved payment methods, and disease control techniques, further including correlating the best practice and the improved payment methods with the value of the cryptocurrency. Element 4, wherein the healthcare network includes, at least partially, a blockchain network including a blockchain database, further including encrypting the skill database in a blockchain string in the blockchain database. Element 5, further including polling a user, a provider, and the skill database for determining a new data event, the new data event including at least one of a new enrollment, a new skill, or a usage activity from the user or the provider. Element 6, further including adding the new skill to the skill database when the correlation coefficient is larger than a pre-selected threshold. Element 7, further including identifying at least one skill in the skill database for which the correlation coefficient is higher than a pre-selected threshold. Element 8, further including identifying when the transaction rate has changed to meet a pre-selected threshold increase, and notifying the financial platform of a new transaction rate. Element 9, further including determining a predicted value of the cryptocurrency when the new skill is added to the skill database, and determining whether to add the new skill to the skill database or not based on the predicted value of the cryptocurrency.

Each of embodiments A, B, and C may also have one or more of the following additional elements in any combination: Element 10, wherein modifying a block string for the skill in the blockchain network includes adding a block with an encrypted secret key in the block string for the skill in the blockchain network. Element 11, wherein the data event includes at least one of usage of a skill by a service provider, addition of a new skill to the skill database, doctor density in an area, or usage of emergency care by at least a portion of the plurality of users, and associating a cryptocurrency value with the data event includes accessing the data event and the cryptocurrency value from a table in the financial platform. Element 12, further including inputting the data event to a machine learning algorithm in the financial platform to determine a predicted cryptocurrency value of the data event, when the data event is a new data event. Element 13, further including providing the third party user with a secured key to access the at least one of the skills from the skill database.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for regulating a value of a cryptocurrency used in a healthcare network, the method comprising:
enabling the healthcare network to connect to a plurality of users and a plurality of service providers;
providing a financial platform responsible for regulating the cryptocurrency used in the healthcare network;
providing a skill database for identifying and storing skills;
determining a correlation coefficient between a transaction rate and a data event, wherein the transaction rate corresponds to transactions made using the cryptocurrency, and the data event comprises at least one of usage of a skill by a service provider, addition of a new skill to the skill database, doctor density in an area, or usage of emergency care by at least a portion of the plurality of users; and
notifying the financial platform for updating the value of the cryptocurrency based on the correlation coefficient.

2. The computer-implemented method of claim 1, wherein the service providers comprise hospitals, insurance companies, Contract Research Organizations (CROs), and drug companies, further comprising adding a new service provider to the healthcare network based on a correlation between the service provider and the value of the cryptocurrency.

3. The computer-implemented method of claims 1 and 2, wherein the skills are machine learned, further comprising adding a new skill based on a correlation between the new skill and the value of the cryptocurrency.

4. The computer-implemented method of claims 1 through 3, wherein the skills comprise a best practice, improved payment methods, and disease control techniques, further comprising correlating the best practice and the improved payment methods with the value of the cryptocurrency.

5. The computer-implemented method of claims 1 through 4, wherein the healthcare network comprises, at least partially, a blockchain network including a blockchain database, further comprising encrypting the skill database in a blockchain string in the blockchain database.

6. The computer-implemented method of claims 1 through 5, further comprising polling a user, a provider, and the skill database for determining a new data event, the new data event comprising at least one of a new enrollment, a new skill, or a usage activity from the user or the provider.

7. The computer-implemented method of claims 1 through 6, further comprising adding the new skill to the skill database when the correlation coefficient is larger than a pre-selected threshold.

8. The computer-implemented method of claims 1 through 7, further comprising identifying at least one skill in the skill database for which the correlation coefficient is higher than a pre-selected threshold.

9. The computer-implemented method of claims 1 through 8, further comprising identifying when the transaction rate has changed to meet a pre-selected threshold increase, and notifying the financial platform of a new transaction rate.

10. The computer-implemented method of claims 1 through 9, further comprising determining a predicted value of the cryptocurrency when the new skill is added to the skill database, and determining whether to add the new skill to the skill database or not based on the predicted value of the cryptocurrency.

11. A system for regulating a value of a cryptocurrency used in a healthcare network, the system comprising:
a memory storing instructions; and
one or more processors configured to execute the instructions to cause the system to:
enable the healthcare network to connect to a plurality of users and a plurality of service providers;
provide a financial platform responsible for regulating the cryptocurrency used in the healthcare network;
provide a skill database for identifying and storing skills;
determine a correlation coefficient between a transaction rate and a data event, wherein the transaction rate corresponds to transactions made using the cryptocurrency, and the data event comprises at least one of usage of a skill by a service provider, addition of a new skill to the skill database, doctor density in an area, or usage of emergency care by at least a portion of the plurality of users; and
notify the financial platform for updating the value of the cryptocurrency based on the correlation coefficient.

12. The system of claim 11, wherein the healthcare network comprises, at least partially, a blockchain network including a blockchain database, and wherein the one or more processors are further configured to encrypt the skill database in a blockchain string in the blockchain database.

13. The system of claims 11 and 12, wherein the one or more processors are further configured to poll a user, a provider, and the skill database to determine a new data event, the new data event comprising at least one of a new enrollment, a new skill, or a usage activity from the user or the provider.

14. The system of claims 11 through 13, wherein the one or more processors are further configured to add the new skill to the skill database when the correlation coefficient is larger than a pre-selected threshold.

15. The system of claims 11 through 14, wherein the one or more processors are further configured to identify at least one skill in the skill database for which the correlation coefficient is higher than a pre-selected threshold.

16. A computer-implemented method for managing transactions in a healthcare network, the method comprising:
enabling the healthcare network to connect to a plurality of users and a plurality of service providers;
accessing a financial platform responsible for regulating a cryptocurrency used in the healthcare network;
accessing, via a blockchain network, a skill database for identifying and storing multiple skills relevant to the healthcare network;
associating a cryptocurrency value with a data event based on an information from the financial platform, the data event involving a third party user accessing at least one of the skills from the skill database;
notifying the financial platform for updating the cryptocurrency value based on the data event; and
modifying a block string for the skill in the blockchain network, based on the data event.

17. The computer-implemented method of claim 16, wherein modifying a block string for the skill in the blockchain network comprises adding a block with an encrypted secret key in the block string for the skill in the blockchain network.

18. The computer-implemented method of claims 16 and 17, wherein the data event comprises at least one of usage of a skill by a service provider, addition of a new skill to the skill database, doctor density in an area, or usage of emergency care by at least a portion of the plurality of users, and associating a cryptocurrency value with the data event comprises accessing the data event and the cryptocurrency value from a table in the financial platform.

19. The computer-implemented method of claims 16 through 18, further comprising inputting the data event to a machine learning algorithm in the financial platform to determine a predicted cryptocurrency value of the data event, when the data event is a new data event.

20. The computer-implemented method of claims 16 through 19, further comprising providing the third party user with a secured key to access the at least one of the skills from the skill database.
